(19) United States
(12) Patent Application Publication
(10) Pub. No.: US 2002/0119928 A1
(43) Pub. Date: Aug. 29, 2002

(54) Dietary supplement compositions

(76) Inventor: Bill H. McAnalley, Grand Prairie, TX (US)

Correspondence Address: CHRISTOPHER J. ROURK, AKIN, GUMP, STRAUSS, HAUER & FELD, L.L.P., P O BOX 688, DALLAS, TX, 75313-0688, US

(21) Appl. No.: 10/001,439

(22) Filed: Oct. 25, 2001

Related U.S. Application Data
(60) Provisional application No. 60/244,029, filed on Oct. 27, 2000.

(51) Int. Cl. A61K 38/40; A61K 31/732; A61K 31/716

(52) U.S. Cl. 514/12; 514/54; 424/535

(57) ABSTRACT

Dietary supplement compositions for promotion and maintenance of good health and immune system support. Defined nutritionally effective amounts of colostrum, lactoferrin, pectin and β-glucan, are used in various inventive compositions as dietary supplements. The dietary supplement compositions may include other non-toxic nutrients including a complex of essential saccharides.

### CROSS-REFERENCE TO RELATED APPLICATIONS

This application claims the benefit of U.S. Provisional Application No. 60/244,029, filed Oct. 27, 2000.

### STATEMENT REGARDING FEDERALLY SPONSORED RESEARCH OR DEVELOPMENT

None.

### REFERENCE TO A MICROFICHE APPENDIX

None.

### BACKGROUND OF THE INVENTION

The present invention pertains to the field of dietary supplement compositions. More specifically, the invention relates to dietary supplement compositions that include colostrum, lactoferrin, β-glucan and in preferred embodiments, citrus pectin.

Colostrum is the first complete food available to newborn mammals and is even essential for life in some mammalian species. Colostrum is the first mammary secretion obtained within the first few hours after birth, and provides many important biological elements and nutrients present in the mother's blood. This pre-milk substance is nature's way of providing protection for the newborn against viruses, bacteria, allergens, and toxins. While the health benefits of colostrum consumption for newborns have been intuitively understood by human cultures throughout time, some cultures have also recognized it as an important food for adults. For example, the Amish once prepared a pudding made from the colostrum produced by the mother of a newborn calf. To this day in India, colostrum is consumed by adults.

Colostrum contains various immune factors, such as lactoferrin (Lf), immunoglobulins (Ig), and proline-rich peptides (PRP), as well as important growth factors for developing healthy muscles and bones. Colostrum, and, particularly, two of its main components, lactoferrin (Lf) and proline-rich peptides (PRP), act to regulate and balance the immune system. Colostrum also activates and regulates numerous immune, growth, and repair processes. This biological activity is not only important in newborns just beginning to live in a hostile environment, but also in adults with over- or under-active immune systems, perhaps resulting from various types of stress. Colostrum and its components exert important biological activities when given to adults, so that its beneficial effects extend well beyond the neonatal period of development. Colostrum and its components also have potent anti-bacterial, anti-viral, and anti-fungal activities to help protect the body from invading pathogens. Colostrum and its growth factors enhance wound healing, cartilage formation, and bone repair, stimulate muscle protein synthesis/inhibit protein breakdown, benefit gastrointestinal (GI) tract structure and function, and act on the immune system to inhibit tissue inflammation and allergic response. The Lf component of colostrum also acts to regulate iron absorption and availability, and has been shown to significantly inhibit tumor development, growth, and metastasis. Colostrum and its components exert their biological activities when given orally, in some cases as a dietary supplement.

Much of the communication of immunologically active substances with the immune system appears to occur via the membranes that line the oral cavity. It is thought that communication with the immune system via the oral mucosa is important in initiating many of the immunomodulatory activities of colostrum. Signals in the form of food, germs, and poisonous substances are initially recognized in the oral cavity, and messages are distributed by a variety of communication pathways to pertinent parts of the body. There are also protective substances that prevent digestion of the active components of orally administered colostrum before they can exert their biological effect(s). The composition of bovine (cow) and human colostrum is similar, and colostrum from both sources is biologically active and indeed exerts similar biological activity in its own as well as other mammalian species when given orally or injected.

The biological activity of fresh, whole colostrum has been examined in newborn animals where its most apparent effects have been shown in calves. Specifically, calves fed colostrum within 1-3 days after birth show increased energy, improved functioning of the gastrointestinal (GI) tract, enhanced efficiency of gamma-globulin absorption, and higher serum levels of insulin-like growth factor-1 (IGF-1), insulin, glucose and albumin compared to calves deprived of colostrum. Prolonged feeding of colostrum to calves stimulates postnatal development of the GI tract by enhancing small intestine villus size. Beneficial effects in the GI tract of humans given colostrum have also been observed. For example, both bovine and human colostrum, given orally, ameliorate diarrhea from GI tract infections in children. In a pilot study, bovine colostrum was also effective in treating diarrhea resulting from Cryptosporidium parvum infection in adult AIDS patients.

Colostrum has been shown to have significant antibacterial activity in vitro. For example, bovine colostrum is bactericidal to Helicobacter pylori. This bacterium is a frequent cause of gastric inflammation in humans. Bovine and human colostrum also inhibit binding of H. pylori to target tissue lipids in vitro. In addition, the cellular component of human colostrum has been shown to phagocytize and kill Escherichia coli and Candida albicans in vitro. Bovine colostrum is also bactericidal to E. coli in vitro. Human colostrum also stimulates B-cell proliferation and antibody secretion in mouse spleen cells, which is also an anti-infection activity.

One of the biological activities of colostrum and its major components is related to activation and regulation of the immune system. In cell culture, human colostrum has been shown to inhibit the proliferation of immune system T cells that have been previously activated, as well as inhibit production of a key immune system cytokine, interleukin-2 (IL-2). Human colostrum has also shown anti-inflammatory activity (such as inhibition of pro-inflammatory enzymes and antioxidant properties) in neutrophil cell cultures and in adult rats injected with human colostrum. These immunosuppressive qualities can help overcome a hyperactive immune system, which can mediate certain autoimmune disorders. Conversely, human colostrum has been shown to activate the immune system by stimulating secretion of cytokines (IL-1, IL-3, and IL-6) by peripheral blood mononuclear cells in vitro. This activation is important when the immune system is needed, for example, to fight infection or cancer.

Various other biological activities for colostrum have been observed in in vitro and in vivo studies, such as its important role in skeletal muscle development. For example, in colostrum-fed neonatal pigs, there is an increase in IGF-1, which markedly increases muscle protein synthesis. Bovine colostrum has also been shown to inhibit muscle protein breakdown in cultured myoblasts, hepatoma cells, and fibroblasts. Other miscellaneous biological activities of whole human colostrum include an antioxidant property, apparently due to a component which is similar to ascorbate and an anti-amoebic property. Bovine colostrum, given orally as a dietary supplement, enhances resistance to intestinal colonization by C. parvum in mice and protects piglets from the clinical effects of pig rotavirus.

One of the immune components of colostrum, secretory IgA (S-IgA), plays a role in protecting against infection. Indeed, an immunoglobulin preparation derived from bovine colostrum that contains IgA reacts against toxins associated with E. coli and Shigella infections in cell cultures in vitro. S-IgA from human colostrum also inhibits adherence of Vibrio cholera to intestinal tissue in vitro, an activity that is consistent with the ability of IgA to prevent bacterial colonization. S-IgA and IgM autoantibodies from human colostrum also protect in vitro against GI autoantigens that can cause autoimmune disease, and thus act to maintain immune homeostasis. Results from three separate clinical studies have shown that oral immunoglobulins from bovine colostrum are anti-diarrheal in adult AIDS/HIV patients suffering from infection by C. parvum, and may account for this same effect reported for whole colostrum. In other biological activities, orally administered S-IgA-enriched anti-measles bovine colostrum has shown a weak effect in multiple sclerosis patients. A follow-up double-blind study, however, found that hyperimmune bovine colostrum provided no symptomatic relief to such patients. S-IgA also inhibits cytokine release in vitro, indicating that it may play an anti-inflammatory role in colostrum. Finally, orally administered immunoglobulins from bovine colostrum are biologically active in other species, such as horses, sheep, deer, and humans.

Growth factors, also present in colostrum, activate various growth and repair processes in the body that are important for proper development, maintenance and healing of GI tract, skeletal muscle and bone structure. For example, transforming growth factor TGF-B from bovine salivary gland and kidney (which is also present in human colostrum) accelerates wound healing in rats when injected daily into wounds in nanogram amounts. TGF-B from bovine bone, which is identical to cartilage inducing factor, induces cartilage formation and bone repair in tissue cultures. TGF-B has also been found to suppress growth of cancer cells (osteogenic sarcoma cells) in vitro. Epidermal growth factor, also found in colostrum, inhibits muscle protein breakdown in cultured myoblasts in vitro.

Another growth factor present in colostrum, insulin-like growth factor (IGF), has been studied with respect to its biological activities. It is found in abundance in bovine colostrum and is structurally similar to IGF in human colostrum. It is most abundant immediately after parturition in human and pig colostrum. It has cytokine-like activities on lymphoid cells of the immune system, and thus may be a natural component of B and T cell lymphopoiesis.

The biological activities of IGF are related to muscle protein metabolism, repair and glucose utilization. For example, IGF stimulates protein synthesis and inhibits muscle protein breakdown in cultured skeletal muscle cells (myoblasts) and hepatoma cells, and in diabetic rats. Injected IGF also appears to partly protect body protein reserves during nitrogen restriction in rats. Fasting or restriction of dietary protein reduces serum IGF in rats in vivo. Increased body weight has also been seen in aged rats when a serum IGF increase was stimulated by growth hormone in vivo. Oral IGF stimulates cell proliferation in the GI tract of newborn pigs. Injected IGF also stimulates wound healing in vivo in rats, and is increased naturally in the healing wound of mice; its increase is delayed in diabetic mice, consistent with the delayed wound healing experienced by diabetics. All of these findings demonstrate that IGF plays a role in the healing process.

In a randomized, placebo-controlled clinical study of insulin-dependent diabetics, subcutaneously injected IGF reduced both the HbAlc levels (a measure of average blood glucose levels over time) and insulin requirements, apparently by suppressing growth hormone activity. This activity may also be due to an IGF-induced increase in glucose utilization, since IGF stimulates glucose transport in human skeletal muscle in vitro.

Serum IGF increases slightly in athletes given oral bovine colostrum supplementation to their diet during training. In this regard, since an adult human produces about 10,000 micrograms (μg)/day of IGF-1, and the range of adult human blood plasma IGF-1 levels is 42-308 μg/liter (L), the low IGF-1 levels present in bovine colostrum dietary supplements would not be expected to significantly increase the normal background concentrations of IGF-1 in blood plasma. For example, it can be calculated from the study cited that athletes who consumed 125 milliliters (ml)/day of bovine colostrum containing approximately 68 μg/L of IGF-1, received approximately 8.5 μg/day of IGF-1 (assuming complete absorption from the GI tract). This compares to normal IGF-1 production of 10,000 μg/day. However, since a slight, but statistically significant, increase in IGF-1 plasma level was noted in the study, the increase may have been due to stimulation of endogenous IGF-1 production by the liver rather than absorption of dietary IGF-1 from the GI tract.

With regard to any possible adverse effects of IGF, it has been observed that levels of renal IGF-1 and IGF binding proteins rise prior to an increase in kidney size in rats with experimentally-induced diabetes; however, no resultant kidney damage was found. It has also been suggested that IGF might contribute to diabetic nephropathy (i.e., kidney disease) by decreasing collagen degradation as seen in diabetic mesangial cells in vitro. However, while this speculation based on in vitro data is theoretically possible, no evidence for nephropathy was found in either of the studies of diabetics or athletes in which IGF was either injected or given orally.

It has further been suggested that IGF-1 is a possible indicator (i.e., biomarker) for certain types of cancer, since its level sometimes increases when certain types of cancer are present. However, using elevated serum levels of IGF-1 as a biomarker for cancer has not been established, since a consistent association with cancer has not been discovered. Since there is no evidence that IGF-1 causes cancer or adversely alters cellular DNA, it appears that increased plasma IGF-1 is a characteristic rather than a cause of the cancer process. This interpretation is consistent with the finding that IGF and IGF receptor binding proteins are increased in tumor cells. Moreover, since IGFs are “growth factors” produced by the liver to provide endocrine support for cell growth and development, IGF-1, like many other growth-promoting substances produced by the body, is involved in tumor cell, as well as normal cell, growth in vitro.

Proline-rich peptide (PRP) is another immunomodulatory component of colostrum. PRP regulates the thymus gland, which produces T-cells that fight viruses and antigens, and plays a key role in balancing an overactive immune system (such as is found in individuals with autoimmune diseases) or an underactive immune system (seen in immuno-compromised individuals); PRP acts both in vitro and in vivo, and is not species specific. PRP has been tested clinically in the treatment of Alzheimer's disease (AD). Orally administered PRP complex from sheep colostrum (100 μg every other day) was found to significantly improve Alzheimer's dementia in a double blind, placebo-controlled study. Psychiatrists blinded to the treatment assignment assessed outcomes. All 15 AD patients stabilized or improved; none of the 31 selenium-treated or placebo control patients improved.

Lactoferrin (Lf) is another component of colostrum. It is also secreted in milk, tears, mucus, and saliva, particularly in response to a pathogen attack. Many of the biological activities of Lf relate to its ability to regulate either the immune system or the availability of iron. Based on studies with bovine and human colostrum evaluated in vitro and/or orally or by injection in test animals or humans, its biological activities include anti-inflammatory, anti-allergenic, anti-cancer, anti-bacterial, anti-viral, and anti-fungal functions. Its anti-inflammatory activity relates to its ability to inhibit pro-inflammatory cytokines, such as tumor necrosis factor-alpha (TNF-a) and IL-6. However, Lf also regulates the immune system by stimulating cytokine release when an activated immune system is needed to, for example, fight infection or cancer.

Its anti-allergenic activity may relate to its ability to inhibit mast cell tryptase, which is involved in bronchoconstriction and airway hyperreactivity. Bovine Lf inhibits the development, growth, and metastasis of tumors in rats and mice when injected or given orally as 0.2% of the diet, perhaps in part by its ability to stimulate natural killer cell release and bind to various glycosaminoglycans. Its anti-pathogenic activities relate primarily to its ability to bind iron and thus starve pathogens, which require iron as a nutrient. Bovine and human Lf also act to prevent colonization by pathogens in vivo by binding to them and preventing their adhesion to host cells. They are particularly effective against H. pylori, which is a common cause of gastric inflammation in humans. Human and bovine Lf have also been shown in vitro to promote the growth of beneficial bifidobacteria.

β-glucan refers to a family of closely related carbohydrates that are made up of long chains of glucose that can be linked and branched in a variety of ways. β-glucans are commonly found in certain mushrooms, baker's yeast, and in the bran of oats and barley, where they may play a role in the cholesterol-lowering properties of brans. β-glucans with a 1,3/1,6 glycosidic linkage have shown particularly strong immunomodulatory activity as biological response modifiers. β-glucan from baker's yeast, which serves as an immune system booster in dietary supplements, is β-1,3/1,6 glucan.

Dietary β-glucan is resistant to digestion, which may be due to the presence of the 1,6-β-D-branched residues of the polysaccharide molecule. There are cellular receptors and plasma binding proteins that are specific for β-glucan which indicate that absorption of intact molecules occurs.

β-glucan acts as a potent regulator of the immune system since it can both stimulate and inhibit various immune system activities. This results in helping the immune system work better without making it too overactive. Animal studies have shown that the immunostimulatory activities of β-glucan can inhibit cancer cell growth and metastasis, and prevent or reduce bacterial infection. The immune system activities of β-glucan appear to depend on its helical conformation. Its large molecular size appears necessary for it to augment multiple immunological activities in vitro. Its biological activities also help to regenerate and repair tissue and modulate inflammation, which aid in wound healing. Human studies have shown that dietary β-glucan lowers blood cholesterol and improves glucose utilization by body cells.

As noted above, β-glucan has biological activity as an immune system regulator. Many of its other biological effects are tied to this activity. The immunoregulatory activities of β-glucan relate mainly to its ability to stimulate or inhibit macrophage release of cytokines involved in immune system control or to modulate macrophage phagocytosis, depending on the needs of the immune system. Thus, it can boost the immune system without overstimulating it.

Numerous studies in animals have shown that β-glucan attracts monocytes, and binds to and activates macrophages to release cytokines. Moreover, differential effects of β-glucan on cytokine production have been observed both in vitro in macrophages and in vivo in mice and appear related to the cross-linking of the β-glucan receptor. In this regard, β-glucans with a 1,3/1,6 glycosidic linkage have shown particularly strong immunomodulatory activity as biological response modifiers.

The immunoregulation of cytokine release by β-glucan is particularly apparent in modulating the adverse response to bacterial sepsis. For example, when rat alveolar macrophages are incubated with β-glucan (>500 μg/mL) and then challenged with bacterial lipopolysaccharide (LPS) in vitro, the expected TNF-α release is significantly suppressed. Additionally, monocytes and lymphocytes isolated from mice injected with β-1,3/1,6 glucan in vivo show suppressed production of proinflammatory cytokines, especially TNF-α, in response to LPS challenge in vitro, which is consistent with an overall decrease in host mortality in the β-glucan-treated mice. β-glucan also inhibits release of the proinflammatory cytokines, interleukin-12, TNF-α, and interferon-gamma by mouse spleen cells stimulated with particulates in vitro. β-glucan also inhibits Candida albicans-stimulated release of arachidonic acid, another inflammatory factor, by monocytes in vitro.

With macrophage phagocytosis, in vitro studies in cell cultures have demonstrated that the phagocytic process is mediated by β-glucan receptors and can be inhibited by β-glucan. Moreover, glucocorticoids, such as dexamethasone or hydrocortisone, have been shown to enhance human monocyte β-glucan receptor activity in vitro, which suggests that endogenous glucocorticoids may play a role in modulating β-glucan-mediated phagocytosis. In another in vitro study, monocyte phagocytosis of C. albicans was also inhibited by β-glucan. However, when β-glucan was injected into mice, it enhanced particulate uptake by peritoneal macrophages, suggesting that it can either inhibit or stimulate the phagocytic process, depending on the needs of the intact immune system.

Accordingly, β-glucan stimulates phagocytosis and release of proinflammatory cytokines when it is necessary to rid tissues of cellular debris, resulting from a wound, or fight a bacterial infection. Additionally, β-glucan has been shown in rats to stimulate the attraction of neutrophils and macrophages to an inflammatory site. However, when tissue inflammation is too severe or phagocytosis might result in too much tissue stress, the process can be inhibited, resulting in modulation of the response. For example, β-glucan has shown immunomodulatory activity in mouse cells in vitro without inducing inflammatory cytokines.

Another biological activity of β-glucan that relates to its ability to stimulate the immune system is its effects on cancer cell growth and tumor development.

Several studies conducted in mice have demonstrated that injected β-glucan stimulates tumor rejection and inhibits tumor growth and metastasis. It has also been shown that the immune system effects for β-glucan are similar in mice and humans, making mouse studies useful models for investigating this activity. β-glucan may exert its anti-tumor effects by activating phagocytosis, enhancing natural killer (NK) cell activity, and stimulating TNF-α, interleukin-6, and interferon release. It has been shown, however, that an excess of β-glucan failed to show anti-tumor activity, apparently due to an inhibition of TNF-α production at the higher level. β-glucan has also been shown to stimulate antibodies to target tumors for cytotoxic recognition by leukocytes and enhance host-mediated anti-tumor activity in vivo. Injected β-glucan actually potentiates the response to chemotherapy in mice. β-glucan also inhibits tumor metastasis. For example, lung metastasis of tumor cells was inhibited in mice injected with β-glucan or given β-glucan orally.

Another biological activity of β-glucan is its ability to help prevent bacterial infection. For example, it can enhance some elements of the immune system against staphylococcal infections when injected in sheep. Mice injected with β-1,3-glucan were protected against pneumococcal infection. β-glucan extracted from oats provides enhanced resistance against Eimeria vermiformis (a pathogenic protozoa) infections in mice when given either orally or by injection. Injected β-glucan has been shown in several separate studies in mice to help prevent bacterial infection following either surgical trauma or radiation-induced immunosuppression.

Yeast derived β-1,3/1,6-glucan enhances leukocyte anti-infective activity in human whole blood in vitro without increasing inflammatory cytokine production. β-glucan also enhances the effectiveness of antibiotic medications against Esherichia coli or Staphylococcus aureus in rats in vivo by enhancing the bactericidal activities of neutrophils and macrophages, again without stimulating proinflammatory cytokine production. In a study in humans, intravenously administered β-glucan enhanced the effectiveness of an anti-fungal drug in ten patients seriously infected with Paracoccidioides brasiliensis. Only one of the ten patients who received the combined therapy relapsed, as compared to five out of eight who received only the anti-fungal drug.

β-glucan has several additional biological activities that generally support body health and well being. For example, dietary supplementation with yeast-derived β-glucan fiber has been shown to effectively lower blood cholesterol in humans. In another study, dietary supplementation with purified oat fiber containing 80% β-glucan resulted in significantly reduced total and LDL cholesterol in hypercholesterolemic adults without changing HDL cholesterol.

In addition to the ability of β-glucan to modulate the inflammatory process, which helps with wound healing, it has also been shown to help regenerate and repair tissue in rats. When given intravenously to rats, the treatment resulted in increased wound tensile strength and collagen biosynthesis. Regarding other biological activities, a human in vitro study showed that β-glucan enhanced hematopoiesis (i.e., blood cell formation). In addition, improved glucose tolerance and utilization has also been demonstrated in humans, when 10% of the diet contained β-glucan.

Certain particulate β-glucan preparations have been shown to act as antigens in mice and in human cells tested in vitro. Injected β-glucan can also result in higher serum immunoglobulin levels. Thus, it is possible that certain β-glucan preparations might have the potential to be allergenic in sensitive individuals. However, this activity is not generally characteristic of sugar polymers taken orally, and β-glucan would be expected to help in decreasing allergy symptoms due to its ability to regulate the immune system and modulate an overactive immune response. One report indicated that injected β-glucan increased the toxicity of orally administered indomethacin in mice.

Pectins are soluble fibers that primarily include soluble chains of 1,4 linked methylated polygalacturonic acid residues found in cell walls from common dietary plants, such as apples, citrus fruits, sugar beets, soy, oat fiber and peas. Pectins may constitute as much as ⅓ of the dry weight of a plant and are also found in some plant juices. The molecular size of pectins generally range from 25,000 to 400,000 Daltons. Commercial citrus pectin is also comprised of a small region (1/40) containing rhamnogalacturonic acid residues. Pectins are generally recognized as safe (GRAS) substances that may be directly added to foods. Citrus pectin in the diet appears to be very biologically active.

The U.S. Food and Drug Administration (FDA) has determined that the average person consumes 0.6-1.0 grams of pectin during an average meal. There is only limited digestion of citrus pectin in the stomach and small intestine in humans. Citrus pectin has been shown in vitro to be the most completely and effectively utilized by animal rumen intestinal bacteria, when compared with apple and sugar beet pectins, and by human fecal bacteria, when compared with soy, sugar beet, pea, and oat fiber pectins. Like most complex, large molecular weight substances from food sources, there are no quantitative absorption data available for citrus pectin, however, its many systemic biological activities are consistent with significant absorption of the intact structure.

One of the biological activities of citrus pectin is its ability to lower blood cholesterol by regulating cholesterol homeostasis and lipoprotein metabolism when given as a dietary supplement to test animals and humans. For example, in guinea pigs fed a high-cholesterol diet supplemented with 7.5-12.5% citrus pectin, LDL cholesterol was reduced by 29-67% compared to animals fed the same diet without citrus pectin supplementation. Citrus pectin was also hypocholesterolemic in guinea pigs fed a diet comprised of 5% citrus pectin plus ascorbic acid. In chickens fed a diet of 2-6% citrus pectin, there was a reduction in blood cholesterol, total lipids, and triglycerides, as well as liver fat and body weight. In fact, there was actually reduced atherosclerosis in aorta and coronary arteries of pigs fed a diet containing 3% grapefruit pectin.

When an amount of citrus pectin equivalent to that present in a high-fiber diet of fruits and vegetables was added to a low-fiber diet in human subjects, blood cholesterol was significantly reduced. In another human study with nine subjects, dietary supplementation with 15 grams of citrus pectin per day for 3 weeks resulted in a 13% decrease in blood cholesterol and increased fecal excretion of fat (44%), neutral steroids (17%), and bile acids (33%), with no change in plasma triglyceride levels.

The hypocholesterolemic effect of citrus pectin may be more than just the result of pectin binding to cholesterol or bile acids. The mechanism may also relate to increased fecal excretion of cholesterol. For example, citrus pectin increased excretion of fecal neutral steroids in rabbits after 5 weeks of a diet containing 10% citrus pectin supplementation. There was also a 6% increase in the excretion of neutral steroids in humans given large amounts (15 grams/day) of citrus pectin for approximately two months. A 14% increase in excretion of cholesterol occurred in six ileostomy patients fed a low-fiber diet supplemented daily with 15 grams of citrus pectin. Also, regression of gallstones occurred in hamsters fed a diet of 4.2% pectin.

Another biological activity of citrus pectin is its ability to inhibit tumor development and metastasis when added to the diets of animals or when tested in vitro. For example, in several separate studies conducted in rats, a diet containing 20% pectin decreased both the number and incidence of colon tumors. In an in vitro study, modified citrus pectin reduced human prostate cancer cell growth as well. Since carcinogen binding to pectin fiber occurs in vitro, it has been suggested that this binding might contribute to inhibition of the colon cancer in rats.

Citrus pectin also appears to inhibit spontaneous metastasis of cancer cells. In one study, rats were injected with prostate cancer cells and fed a diet containing 0.1-1.0% citrus pectin. At 30 days post cancer-cell injection, 7/14 rats in the 0.1% and 9/16 rats in the 1% treatment groups had lung metastases compared with 15/16 control animals. Furthermore, based on in vitro binding experiments, citrus pectin inhibited prostate cancer cell adhesion to rat endothelial cells by competing for galactose-binding lectins (gal-lectins) on the surface of the cells, essentially acting as a cancer cell “decoy”. In a separate study in mice, injected citrus pectin was also shown to inhibit melanoma cell metastasis to the lungs by competing for gal-lectin binding sites on the tumor cells.

A large body of scientific evidence supports the health benefits of soluble fiber consumption. Citrus pectin in the diet appears to modulate the structure and function of the gastrointestinal (GI) tract, and enhances its healing following bowel surgery or inflammation. For example, a 14-day diet containing 2.5% pectin induced a significant increase in the villus height and crypt depth in the small intestine of rats, and increased the number of goblet cells in the intestinal epithelium for chicks fed a diet containing 3-4% pectin. There was also improved bowel mucosal structure, prolonged intestinal transit, and decreased diarrhea in rats fed a diet containing 2% pectin. Pectin has shown an immunoregulatory effect on the intestinal immune system by increasing serum IgA and IgG in rats fed a diet containing 5% pectin.

Citrus pectin also promotes healing of the colon following colonic surgery in rats fed a diet containingl % pectin. The improved healing may be a local effect mediated by the presence of short-chain fatty acids resulting from the fermentation of pectin. A diet containing 1% pectin has also been shown to facilitate intestinal recovery from enterocolitis in rats.

Dietary pectin appears to improve glucose utilization by the body and modulate weight gain and body fat. For example, increases in blood glucose levels after eating a meal were lower when 10 grams of pectin was added to the diet of non-insulin dependent diabetics. In addition, although daily weight gain was increased in pigs fed a diet containing 4% citrus pectin, body fat was reduced. Citrus pectin actually depressed weight gain in chickens, suggesting a possible differential effect on body weight.

Since pectins have been shown to bind heavy metals, such as lead, copper, cobalt, and nickel, in vitro, citrus pectin may help remove toxic contaminants from biological systems. One study has also demonstrated increased absorption of certain (calcium, magnesium, manganese, zinc, and copper), but not all (iron) minerals and nutrient ions, in rats fed citrus pectin for 6 weeks. Also, ingestion of 14 grams of citrus pectin by human subjects taking the drug valproic acid did not result in any significant effect on GI absorption of the drug.

Citrus pectin may affect and indeed inhibit β-carotene utilization. In a human study, when 12 grams of citrus pectin was added to a meal supplemented with 25 mg β-carotene, plasma β-carotene levels were reduced by >50%. In another study of chicks, a diet containing 7% citrus pectin resulted in depressed β-carotene utilization, when compared with animals not receiving citrus pectin supplementation.

Although citrus allergies are uncommon, there is limited evidence to suggest that citrus pectin may have the potential to be allergenic in sensitive individuals. This is not surprising, since many food substances have the potential to act as antigens. In any event, the dietary implications of such findings are unclear, since balanced diets should contain significant and adequate amounts of both pectin and β-carotene supplied from fruits and vegetables.

### SUMMARY OF THE INVENTION

The present invention relates to dietary supplement compositions comprising a combination of colostrum, lactoferrin and β-glucan, thus providing an improvement over previous compositions which lacked one or more of these components. When absorbed in combination, the effects of colostrum, lactoferrin and β-glucan on the health and well-being of the recipient are surprisingly beneficial, and include: promotion of immune system health, promotion of body health and athletic performance, promotion of gastrointestinal (GI) tract health, promotion of blood vessel health, promotion of glucose utilization and blood sugar balance, improved pathogen resistance, improved cancer inhibition and improved mental function and toxin-related activities. Absorption of the components of the compositions of the present invention in the oral cavity, rather than through the lining of either the stomach or intestine is particularly efficacious, therefore the present invention includes the provision of the components of the inventive composition in a mucosal delivery format as a chewable product.

The present invention provides dietary supplement compositions comprising colostrum, lactoferrin and β-glucan, and preferably may also include citrus pectin. The present invention also provides compositions containing these ingredients which may also include nutritionally acceptable carriers, diluents and flavorings, a method of administering such a composition in a form appropriate for absorption through the lining of the oral cavity, and a method of stimulating immune function.

### DETAILED DESCRIPTION

The compositions of the present invention provide support for and promote strong immune systems. In a preferred embodiment, the compositions of the present invention are provided in a chewable delivery system and feature a targeted array of nutrients for building and nurturing immune health, especially when stress tends to bear down on the immune system. The preferred chewable delivery system of the compositions of the present invention optimizes the first-line defenses of the mucosal immune system. The oral cavity is the gate of entry to the immune system. The oral cavity mucus membrane cell receptors are activated by immunoreactive ingredients and communicate with the lymphatics, where activated lymphocytes spread signals to the blood and other tissues. The oral cavity epithelial cells and GI tract cells are sites for systemic absorption of smaller sized immunoreactive ingredients. Also, immunoreactive ingredients react directly with bacteria, viruses, and fungi in the oral cavity and esophagus.

Those of ordinary skill in the art will recognize that supporting the mucus membranes that line the mouth, nose, lungs and digestive tract is beneficial since the majority of harmful invaders of the body must pass through these defenses. These membranes comprise the largest barrier in the body and form the first line of defense. When the mucosal immune response is triggered, it makes natural antibodies that “arm” saliva, tears, bronchial and nasal secretions, as well as fluids in the digestive tract, turning them into the body's own powerful, natural antimicrobial wash. The preferred chewable delivery system enhances the ability of the nutrients in the compositions of the present invention to immediately react with mucus membranes in the mouth and esophagus to energize the immune system and begin the fight against bacteria. In addition, the uptake and immune system activities of nutrients from the compositions of the present invention begin in the mouth as they are chewed and continue in a cascade of immune responses throughout the immune system. The components of the compositions of the present invention bind to specific receptor sites that send signals directly to the lymphatic system—the roadway for the approximately trillion circulating white blood cells on patrol against invaders. The nutrient forces in the compositions of the present invention spread throughout the gastrointestinal (GI) tract where they facilitate normal cell proliferation, growth of beneficial GI bacteria, and discourage growth of bad bacteria. Those of ordinary skill in the art will recognize that the lymphatic tissue in the GI tract is one of the major subdivisions of the immune system. Everyday stress such as work, traffic, and taking care of a family can accelerate the body's use of nutrients. A poor diet and sleep deprivation place additional demands on and challenge the immune system.

The job of the immune system is to recognize and eliminate foreign particles and organisms and maintain balance in every part of the body. With the right nourishment, the immune system performs these functions quite admirably. Under stress, however, the immune system has to work harder. Because stress acts as a drain on nutrients, more nutritional support is needed for the immune system during periods of stress. Also, if the immune system is unbalanced, it needs more nutritional power to assist in its efforts to compensate and achieve balance.

When the immune defenses are weakened and unprepared, unwanted viruses work around system failures, microbes make themselves at home and toxins take hold. Emotional stress is a silent threat to good health. While the signs of emotional stress may be recognized, one often fails to acknowledge the burden it places on the body. In addition, even when one knows they are physically stressed, whether from work or a workout, one often thinks that all that is needed is rest. But that may not be all the immune system needs to bounce back. In addition, one may fail to recognize the environmental stress the body incurs that can make the immune system work harder, or the dietary stress caused by making poor food choices. To help combat environmental stress the compositions of the present invention support the body's ability to conduct cellular “house cleaning” by helping to remove heavy metals, toxins and damaged cellular material from cells.

To help the body handle physical stress, the compositions of the present invention include peptides, amino acids and glycoproteins that engage the body's natural ability to heal and repair itself.

The components of the compositions of the present invention help trigger a cascade of immune signaling mechanisms. This is especially important when stress factors work against the immune system. Glyconutrients help carry these immune signals to other cells. According to a preferred embodiment, the compositions of the present invention are combined with a complex of essential saccharides such as the dietary supplement sold by Mannatech Inc. of Coppell, Texas under the trade name “Ambrotose®” The Ambrotose® product is preferably produced according to the methods and procedures set forth in International Patent Application Publication Number WO 98/06418, the entire disclosure of which is hereby incorporated by reference herein. The combination of the compositions of the present invention and Ambrotose® complex provide a synergistic array of proteins, peptides, polypeptides, and glyco-proteins-nutrients that can help to achieve optimal health through an appropriately immunomodulated immune system.

The compositions of the present invention preferably include ingredients that promote the production of new blood cells, cartilage formation and bone growth.

The compositions of the present invention enhance muscle protein synthesis and insulin-like growth factor release in tissues. The compositions of the present invention also assist the body in regulating muscle protein breakdown and bio-regulation during stress.

The present invention encompasses compositions, dietary supplements and methods for their use that include the following active components:

(a) Prime colostrum has the highest concentrations of immunoglobulins, interferons, proline-rich peptides, amino acids and vital enzymes produced by mammary tissue, being higher than those produced in ordinary colostrum. This provides the newborn with protection against viruses and bacteria and other health threats. Besides providing the first complete food for the newborn, prime colostrum has such profound immuno-stimulating properties that administration of very small amounts activates the human immune system. In addition to this immune stimulation capability, prime colostrum provides immunoglobulins directly (e.g. IgA, IgG, IgM) and also growth factors (e.g. IGF-I, TGF A and B). It supplies immunomodulatory proline-rich peptides which moderate the activity of the immune system through their effect upon the thymus gland, stimulating under-active immune systems such as those of immuno-compromised persons, or moderating those that are over-active as in individuals with auto-immune diseases. Prime colostrum slows muscle breakdown, improves protein synthesis and utilization, provides digestive enzymes, regulates blood sugar and stimulates growth and repair. In sum, prime colostrum contains powerful healing, growth and repair factors that activate numerous immune, healing, growth and repair systems and assist in synthesis, retention and repair of muscle, bone, nerve and cartilage. As the body ages, becomes weakened by illness, or is subjected to physical stresses, it produces less and less of the factors that are needed to overcome metabolic insults or infection and to heal quickly. See Canadian Patent Application No. 2,279,791, the entire disclosure of which is hereby incorporated herein by reference.

(b) Lactoferrin is an iron binding protein that occurs naturally in the body. It is secreted in milk, tears and saliva, and is expressed by white blood cells. Lactoferrin is well known in the art as a biological regulator that performs many important functions in the body. These functions include maintaining a healthy balance in the digestive tract, helping the immune system and promoting healthy cell growth. Dairy cattle provide a cost-effective source of lactoferrin for inclusion into a dietary supplement. Lactoferrin from cows' milk can be prepared free of lactose. Lactoferrin bioregulates iron, boosts the immune system, balances the digestive tract, increases energy and stamina, and promotes cell growth and healing. These broad, beneficial properties are surprising in view of the inability of bovine lactoferrin to bind to the lactoferrin receptors at the surface of the mucosal cells of human small intestine.

(c) Citrus pectin is a preferred component that endows the composition with additional benefits as a nutritional supplement. Citrus pectin is a protein that contains galactose molecules on its surface which are able to bind lectins involved in the transmission within the body of certain types of cancer.

(d) Citric acid is a preferred component that may be incorporated to promote salivation and to adjust the acidity of the composition in order that solubility, activity and absorption of the components within the oral cavity is enhanced.

(e) The β-glucan incorporated in the compositions of the present invention is disclosed in U.S. Pat. Nos. 5,223,491, 5,397,773, 5,519,009, 5,576,015, 5,702,719, 5,705,184, the entire disclosures of which are hereby incorporated herein by reference. Glucans are polymers of glucose. Such glucans may be derived from the cell walls of yeast. Glucan extracted from yeast is a potent stimulator of the immune system.

Iron is a key mineral required by all microorganisms for maintenance and growth. Excess iron in the intestines promotes pathogen growth and proliferation. Lactoferrin from cows' milk is partially saturated with iron (approximately 25% of total saturation) providing a dietary source of iron as well as a means of scavenging free iron from the oral cavity and digestive tract. Lactoferrin works on contact to starve pathogens of iron so that the correct balance of beneficial bacteria develops and is maintained in the digestive tract. The growth of harmful bacteria that are poorly adapted to these conditions is inhibited. By sequestering iron and delivering it for use by the cells of the body's internal tissues, lactoferrin improves digestion and boosts the body's natural defense mechanisms. This generates more energy and increased stamina for physical activities and optimum health.

Lactoferrin and prime colostrum achieve their optimal effects when dissolved slowly in the mouth, rather than being swallowed directly in the form of a pill or capsule. Slowly dissolving the lactoferrin and constituents of colostrum in the mouth permits their absorption into the capillaries at the surface of the oral cavity's lining, which is able to occur before the lactoferrin and prime colostrum are exposed to the harsh degradatory conditions of the stomach and intestines. For example, bovine lactoferrin is less resistant to degradation in the human digestive tract than is human lactoferrin, and the lactoferrin receptors in the small intestine of humans will not bind bovine lactoferrin. Thus administration of bovine lactoferrin to humans in a mucosal delivery format, such as a format that enables its absorption through the lining of the mouth, is particularly efficacious. Immunoglobulins from colostrum also pass directly into the blood through the inner mucosal layer of the mouth. Orally delivered prime colostrum stimulates the body to replace growth, healing and repair factors as needed and produce them naturally to achieve homeostasis. Oral administration of citrus pectin has been shown to be effective for inhibiting spontaneous metastasis of a rat prostate cancer.

Chewable tablets, in contrast to pills or capsules, provide a ‘mucosal delivery format’ (MDF) for constituents which can be absorbed through the oral mucosal surface, such as the colostrum, lactoferrin or citrus pectin of the compositions of the present invention. In particular, the chewable tablets of the present invention are able to enhance the benefits associated with absorption of appropriate constituents through the oral epithelial mucosa and into the underlying lymphatic system, for they are designed to be chewed in the mouth; such tablets are therefore a preferred MDF.

The compositions of the present invention provide a nutritional and dietary supplement for immune support that features a chewable formula containing: colostrum, lactoferrin, 1-3/1-6 glucan; and citrus pectin. The individual components of the composition may be obtained from commercial sources: colostrum (which is dehydrated by standard spray-drying procedures known in the art) from any processing facility approved by the United States Food and Drug Authority (FDA) such as Immuno-Dynamics, Inc. of Perry, Iowa under the trade name “Prime Colostrum®”; lactoferrin from approved manufacturers such as DMV International Nutritionals of Frazier, N.Y.; the 1-3/1-6 glucan is commercially available from Biopolymer Engineering, Inc. under the tradename “Beta Right™,” citrus pectin from approved distributors or manufacturers such as G.C.I. of Los Angeles, Calif.; flavors from approved distributors or manufacturers such as Allen Flavors, Inc. of Edison, N.J. Manufacturing of the composition, the dietary supplement, and the oral dosage forms may each be performed using standard techniques well known to those of ordinary skill in the art which are appropriate for the food or pharmaceutical industries, such as at any FDA approved facility.

Key Immune Health Benefits of the Compositions of the Present Invention:

Fast-acting.

Energizes a cascade of immune responses that begin in the mouth and proceed throughout the body.

Optimizes response of natural killer cells, B-cells and T-cells which seek out and destroy foreign substances.

Reacts with the specific cell receptors that cause the cells to engulf and destroy bacteria and cellular debris.

Strengthens the mucosal immune system, your first-line defense.

Supplies and enhances natural antibodies (immunoglobulins).

Helps regulate the number and activities of circulating immune cells.

Initiates communication in the immune system which releases chemical messengers to fight infection and disease.

Supports immune cell growth and proliferation in the GI tract.

Binds iron so that it starves bad bacteria throughout the body—re-routing it to become more bio-available for beneficial uses.

Helps the body remove heavy metals and toxins in the cells.

Contains proteins that are combined with saccharides to make glycoproteins for hormones and enzymes used in immune regulation.

Helps balance the immune system.

Table 1 below lists some of the biological activities of various components of the compositions of the present invention.

TABLE 1
BIOLOGICAL ACTIVITIES OF VARIOUS COMPONENTS
OF THE COMPOSITIONS OF THE PRESENT INVENTION

| | | |
|---|---|---|
| Biological Activity | Ingredient | Data Source |

Immune system health

| | | |
|---|---|---|
| Regulates the immune system | PRP, Col, Lf, | human/animal |
| | β-Glu, P | |
| Inhibits cytokine release | Lf | human/in vitro |
| Stimulates cytokine release | Col | in vitro |
| Prevent autoimmune response from | S-IgA, Col, Lf | animal/in vitro |
| intestinal pathogens | | |
| Promotes phagocytosis by neutrophils | Lf | in vitro |
| Stimulates B-cell and antibody secretion | Col | in vitro |
| Inhibits mast cell enzyme involved in | Lf | animal |
| allergic airway response | | |
| Enhance NK cell activity | Lf | animal |

Body health and athletic performance

| | | |
|---|---|---|
| Stimulates muscle protein synthesis | IGF | animal |
| Inhibits muscle protein breakdown | IGF, EGF, Col | animal/in vitro |
| Stimulates wound healing/tissue repair | IGF | animal |
| Induces cartilage formation and bone | TGF-β | in vitro |
| repair | | |
| Anti-inflammatory properties | S-IgA, Col, Lf | animal |
| Bioregulation during trauma stress | Lf | human |
| Enhances hematopoietic activity | β-Glu | in vitro |
| Increases IGF in tissues | Col | human/animal |

Gastro-intestinal (GI) tract health

| | | |
|---|---|---|
| Anti-diarrhea from GI tract infection | Col, Ig | human |
| Stimulates GI tract growth/development | IGF, Col, P | animal |
| Improves normal functioning of GI tract | Col, P | animal |
| Promotes growth of beneficial GI | Lf | in vitro |
| bacteria | | |

Blood vessel health

| | | |
|---|---|---|
| Lowers blood cholesterol | β-Glu, P | human |
| Enhances bile acid/cholesterol excretion | P | human |
| Reduces atherosclerosis | P | animal |

Glucose utilization and blood sugar balance

| | | |
|---|---|---|
| Improves glucose tolerance | β-Glu, P | human |
| Reduces average blood glucose (A1c) in | IGF | human |
| non-insulin dependent diabetics | | |
| Stimulates glucose uptake by muscle | IGF | Animal |

Pathogen-related activities

| | | |
|---|---|---|
| Inhibits binding of bacteria to host | S-IgA, Col, Lf, | animal/in vitro |
| tissue | β-Glu | |
| Inhibits growth/kills bacteria | Col, Lf | in vitro |
| Protects against/kills viruses | Lf | in vitro |
| Enhances activity of antibiotics | β-Glu | animal/in vitro |
| Anti-fungal properties | Lf | in vitro |
| Anti-amoebic properties | Col | in vitro |

Cancer-related activities

| | | |
|---|---|---|
| Prevents tumor development | Lf, β-Glu, P | animal |
| Inhibits tumor cell growth | Lf, β-Glu, P, | animal/in vitro |
| | TGF-β | |
| Inhibits tumor metastasis | Lf, β-Glu, P | animal |
| Enhances NK cell toxicity to tumor | Lf, β-Glu | in vitro |

Mental function and toxin-related activities

| | | |
|---|---|---|
| Improves Alzheimer's dementia | PRP | human |
| Binds heavy metals | P | in vitro |
| Anti-oxidant properties | Col | in vitro |
| Reacts against bacterial toxins | Ig | in vitro |

Key: Col = colostrum, PRP = praline-rich peptide, S-IgA = secretory immunoglobulin A, Lf = lactoferrin, β-Glu = beta-glucan, P = pectin, IGF = insulin-like growth factor, TGF = transforming growth factor, EGF = epidermal growth factor, Ig = immunoglobulins

Preferred embodiments of the invention include compositions and dietary supplements, as described above, prepared in a ‘mucosal delivery format.’ A particularly preferred embodiment is an oral dosage form that promotes absorption of the dietary supplement's components through the epithelial lining of the oral cavity. Examples of oral dosage forms that promote absorption of the dietary supplement's components within the oral cavity are those that either encourage retention of the dose within the oral cavity for an extended period, or discourage swallowing of the dose. Such exemplary oral dosage forms include those that are chewable, are appropriate for sucking, and/or encourage salivation, for example, lozenges, particularly chewable lozenges, chewable tablets and chewable gums. The addition of natural or artificial flavouring also encourages retention of the dosage form within the mouth, particularly with children, so that there is greater transfer of the active components through the lining of the oral cavity and into the bloodstream and/or the lymphatic system. Such active components include the constituents of colostrum and the lactoferrin, as described above. The physical size and consistency of the dosage form may also be adapted to prevent premature swallowing of the delivered dose. A preferred period for which the dose should remain in the mouth for effective absorption is 30 seconds to 10 minutes, with better effects being observed at the longer retention times. Larger chewable forms are appropriate for animals that would otherwise be likely to swallow such foodstuff with little mastication.

Further preferred embodiments are methods for promoting those beneficial effects in mammals described above, in which such oral dosage forms of these compositions and dietary supplements are administered.

Exemplary formulations for the dietary supplement of the present invention are described in the following examples. Weight percentages indicated for each ingredient are percentages of the total weight of the end product.

### EXAMPLE 1

In a preferred embodiment of the present invention, the composition comprises the following ingredients cold pressed into a chewable lozenge of hardness 14 to 44 Kp that is taken as a nutritional supplement one to five times per day: 150 mg to 200 mg bovine prime colostrum (which is about 5 to about 40 weight percent), 10 mg to 20 mg bovine lactoferrin (which is about 0.333 to about 4 weight percent), 5 mg modified citrus pectin (which is about 0.167 to about 1 weight percent), 1295 mg to 1945 mg dextrose (which is about 43.2 to about 64.8 weight percent), 7.5 mg to 12.0 mg citric acid (which is about 0.25 to about 2.4 weight percent), 4.5 to 15.0 mg natural and/or artificial flavour (which is about 0.15 to about 0.5 weight percent), 7.5 mg silicon dioxide (which is about 0.25 to about 1.5 weight percent), and 7.5 mg magnesium stearate and dextrose (which is about 0.25 to about 1.5 weight percent), to a total weight of 0.5 to 3.0 grams. The lozenge is chewed for 30 seconds to ten minutes to maximize absorption of the active ingredients through the lining of the oral cavity and their absorption into the blood and lymphatic system.

### EXAMPLE 2

In a preferred embodiment of the invention, each of the following ingredients is placed, in powdered form, into a commercial mixer: 150 parts bovine prime colostrum, (about 10 weight percent), 10 parts bovine lactoferrin (about 0.667 weight percent), 5 parts modified citrus pectin (about 0.333 weight percent), 1297.5 parts dextrose (about 86.5 weight percent), 7.5 parts citric acid (about 0.5 weight percent), 15 parts natural strawberry flavor (about 1.0 weight percent), 7.5 parts silicon dioxide (about 0.5 weight percent) and 7.5 parts magnesium stearate (about 0.5 weight percent). If necessary, the materials are passed through a # 10-12 mesh screen to remove aggregates. Each of the procedures should be performed with precautions against exposure to the powders and dusts that are formed, and particularly against their inhalation. After 20 minutes of thorough mixing, cold pressing the composition in a tablet press set at a maximum pressure of 6.4 tons yields lozenges of weight 1500 mg and hardness 34 to 36 Kp.

### EXAMPLE 3

In another preferred embodiment of the invention, each of the following ingredients is placed, in powdered form, into a commercial mixer following the same procedure as for Example 2: 200 parts (about 9.09 weight percent) bovine prime colostrum, 20 parts (about 0.909 weight percent) bovine lactoferrin, 5 parts (about 0.227 weight percent) modified citrus pectin, 1943.5 parts (about 88.3 weight percent) dextrose, 12 parts (about 0.545 weight percent) citric acid, 3 parts (about 0.136 weight percent) natural strawberry flavor, 1.5 parts artificial flavor (e.g. vanilla, chocolate) (about 0.068 weight percent), 7.5 parts (about 0.341 weight percent) silicon dioxide and 7.5 parts (about 0.341 weight percent) magnesium stearate. After mixing and cold pressing as in Example 2, lozenges of weight 2200 mg were formed which demonstrated a hardness of 34 to 36 Kp.

### EXAMPLE 4

Each of the following ingredients is placed, in powdered form, into a commercial mixer following the same procedure as for Example 2: 200 parts (about 33.3 to about 50 weight percent) bovine prime colostrum, 20 parts (about 3.33 to about 5.0 weight percent) bovine lactoferrin, 5 parts (about 0.833 to about 1.25 weight percent) modified citrus pectin, approximately 215 parts (about 35.8 to about 53.8 weight percent) dextrose and/or maltodextrin, approximately 10 parts stearic acid as binder (about 1.67 to about 2.5 weight percent). After mixing and cold pressing as in Example 2, lozenges of total weight 400 mg to 600 mg were formed.

### EXAMPLE 5

In another preferred embodiment, the invention comprises a composition in which the lactoferrin is present at a concentration of from about 10 mg to about 100 mg per 1500 mg total weight (which is about 0.667 to about 6.67% total weight) and colostrum, present at a concentration of from about 125 mg to about 1250 mg per 1500 mg total weight (which is about 8.33% to about 83.3% total weight). In such a preferred embodiment, a 1500 mg dose is typically provided from one to about five times per day. Supplementary doses may be warranted under particular nutritional or physiological conditions. Additional preferred embodiments include such compositions for use as a dietary supplement that additionally comprise modified citrus pectin at a concentration of from about 1.5 mg to about 15 mg per dose (which is about 0.100 to about 1.00% total weight).

Generally, the effective amount of glucan in a dietary composition prepared according to the present invention will preferably range from about 0.001 w/w % to about 10 w/w % of the composition, more preferably from about 0.1 w/w % to about 4 w/w %. An exemplary formulation for the dietary supplement of the present invention comprising β-glucan is as follows:

### EXAMPLE 6

A preferred embodiment of the dietary supplement of the present invention comprising β-glucan is as follows:

| | Mg/Tablet | Ingredient | Weight % |
|---|---|---|---|
| | 9.7500 | Citric Acid | 0.626 |
| | 1297.0000 | Dextrose | 83.300 |
| | 7.5000 | Magnesium Stearate | 0.482 |
| | 7.5000 | Silicon Dioxide | 0.482 |
| | 30.0000 | Stearic Acid | 1.930 |
| | 5.0000 | Citrus Pectin | 0.321 |
| | 10.0000 | Lactoferrin | 0.642 |
| | 20.4000 | Strawberry Natural Flavoring | 1.310 |
| | 150.0000 | Colostrum | 9.630 |
| | 20.0000 | β-Glucan | 1.280 |
| Total: | 1,557.1500 | | 100 |

According to the foregoing examples, dietary supplements prepared according to the present invention comprise β-Glucan in an amount from about 0.001 to about 10 weight percent, and one or more of colostrum, in an amount from about 5 to about 83.3 weight percent, lactoferrin, in an amount from about 0.909 to about 6.67 weight percent, and citrus pectin, in an amount from about 0.1 to about 1.25 weight percent. Preferred embodiments further comprise citric acid, in an amount from about 0.25 to about 2.4 weight percent, dextrose, in an amount from about 35.8 to about 88.3 weight percent, magnesium stearate, in an amount from about 0.25 to about 1.5 weight percent, silicon dioxide, in an amount from about 0.25 to about 1.5 weight percent, stearic acid, in an amount from about 1.67 to about 2.5 weight percent, and a flavoring, in amount from about 0.15 to about 1.31 weight percent.

Following below is a certificate of analysis of the Colostrum product sold by Immuno-Dynamics, Inc. of Perry, Iowa:

CERTIFICATE OF ANALYSIS
Lot #555 First Milking Bovine Colostrum Powder

| | |
|---|---|
| Protein (N × 6.38) | 53.4% |
| IgG (V.M.R.D., Pullman, WA) | 20.4% = 38.2% of protein |
| Total Immunoglobulin | 23.5% = 44.0% of protein |
| IgG (Bethyl Labs, Montgomery, TX | 15.7% |
| Fat | 21.4% |
| Lactose | 9.5% |
| Moisture | 3.1% |
| Total Coliforms | <3 cfu/g presumptive |
| Salmonella | Neg./25 g presumptive |
| Lead | <1 ppm |
| Nickel | <1 ppm |
| Arsenic | <0.4 ppm |
| Mercury | <0.20 ppm |
| Other Typical Analysis | |
| Physical: | |
| Color | white/cream |
| Taste/Odor | creamy milk |
| Bulk Density (tapped) | 350 g/l |
| Solubility | 200 g/l |
| Chemical: | |
| pH (10% solution @ 20 C.) | 6.4 |
| Ash | 4.5% |
| Microbiological: | |
| Standard Plate Count | <3,000 cfu/g |
| Coliform Count | Neg./25 g |
| E. Coli | Neg./25 g |
| Salmonella sp. | Neg./25 g |
| Mold & Yeast | Neg./25 g |
| Mycobacterium bovis | Negative |
| Mycobacterium avis | Negative |
| M. paratuberculosis | Negative |
| Storage: | Cool, dry area away from sunlight |
| Hazard Data: | Food substance; no known |
| | toxicities or overdoses |
| Special handling: | None; food substance |
| Ventilation: | No special requirements |

Those of ordinary skill in the art will recognize that the foregoing examples are exemplary of the dietary supplement compositions, and that the capsule size, specific amount of each ingredient, and the combination of ingredients can be varied as needed. The dietary supplement compositions of the present invention are preferably administered via the oral mucosal system.

The above is a detailed description of particular embodiments of the invention. Those of ordinary skill in the art should, in light of the present disclosure, appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more”. The phrase “substantially the same” is taken to mean that a first amount, or property, is about 90%-110%, preferably 95%-105%, or more preferably 99%-101%, of the value of a second amount, or property, respectively. The terms “substantially” or “substantial” are taken to mean a “major portion”, “more than 50%”, preferably “more than 90%”, or more preferably “more than 95%” of a particular amount or property.

What is claimed is:

1. A dietary supplement composition for a mammal, comprising a nutritionally effective amount of β-glucan and colostrum.

2. The dietary supplement composition of claim 1, further comprising a nutritionally effective amount of lactoferrin.

3. The dietary supplement composition of claim 1, further comprising a nutritionally effective amount of citrus pectin.

4. The dietary supplement composition of claim 3, further comprising a nutritionally effective amount of lactoferrin.

5. A dietary supplement composition for a mammal, comprising a nutritionally effective amount of β-glucan and lactoferrin.

6. The dietary supplement composition of claim 5, further comprising a nutritionally effective amount of citrus pectin.

7. A dietary supplement composition for a mammal, comprising a nutritionally effective amount of β-glucan and pectin.

8. The dietary supplement composition of claim 1 wherein said mammal is a human.

9. The dietary supplement composition of claim 4 wherein said composition comprises ftom about 5 to about 83.3 weight percent of said colostrum, from about 0.909 to about 6.67 weight percent of said lactoferrin, from about 0.1 to about 1.25 weight percent of said citrus pectin, and from about 0.001 to about 10 weight percent of said β-glucan.

10. The dietary supplement composition of claim 4 further comprising a nutritionally effective amount of citric acid.

11. The dietary supplement composition of claim 10, wherein said composition comprises from about 0.25 to about 2.4 weight percent of said citric acid.

12. The dietary supplement composition of claim 4 further comprising a nutritionally effective amount of citric acid, dextrose, magnesium stearate, silicon dioxide and stearic acid.

13. The dietary supplement composition of claim 12, wherein said composition comprises from about 0.25 to about 2.4 weight percent of said citric acid, from about 35.8 to about 88.3 weight percent of said dextrose, from about 0.25 to about 1.5 weight percent of said magnesium stearate, from about 0.25 to about 1.5 weight percent of said silicon dioxide, and about 1.67 to about 2.5 weight percent of said stearic acid.

14. The dietary supplement composition of claim 12, further comprising one or more of a nutritionally acceptable carrier, diluent or flavoring.

15. The dietary supplement composition of claim 13, further comprising a flavoring in an amount of about 0.15 to about 1.31 weight percent.

16. The dietary supplement composition of claim 4 wherein said composition is prepared in a chewable delivery system.

17. The dietary supplement composition of claim 14 wherein said composition comprises about 9.63 weight percent of said colostrum, about 0.642 weight percent of said lactoferrin, about 0.321 weight percent of said citrus pectin, about 1.28 weight percent of said β-glucan, about 0.626 weight percent of said citric acid, about 83.3 weight percent of said dextrose, about 0.482 weight percent of said magnesium stearate, about 0.482 weight percent of said silicon dioxide, about 1.93 weight percent of said stearic acid, and about 1.31 weight percent of said nutritionally acceptable carrier, diluent, or flavoring.

18. The dietary supplement composition of claim 4 further comprising a complex of essential saccharides.

19. The dietary supplement composition of claim 18, wherein the complex of essential saccharides comprises saccharides provided in oligomeric or polymeric forms as found in: gum tragacanth, guar gum, grain flour, rice flour, sugar cane, beet sugar, potato, milk, agar, algin, locust bean gum, psyllium, karaya gum, seed gums, Larch tree extract, aloe vera extract, gum ghatti, starch, cellulose, degraded cellulose, fructose, high fructose corn syrup, pectin, chitin, acacia, gum arabic, alginic acid, carrageenan, dextran, xanthan gum, chondroitin sulfate, sucrose, acetylated polymannose, maltose, glucan, lentinan, mannan, levan, hemi-cellulose, inulin, fructan, and lactose.

20. A dietary supplement composition for producing in a mammal a first effect selected from the group consisting of regulation of the immune system, regulation of cytokine release, prevention of autoimmune response from intestinal pathogens, promotion of phagocytosis by neutrophils, stimulation of B cell and antibody secretion, inhibition of mast cell enzyme involved in allergic airway response, enhancement of natural killer cell activity, stimulation of muscle protein synthesis, inhibition of muscle protein breakdown, stimulation of wound healing, stimulation of tissue repair, induction of cartilage formation and bone repair, anti-inflammatory effects, bioregulation during trauma stress, enhancement of hematopoietic activity, increase in insulin-like growth factor in tissues, antidiarrheal effect on gastrointestinal tract infection, stimulation of gastrointestinal tract growth, improvement in function of the gastrointestinal tract, promotion of the growth of beneficial gastrointestinal bacteria, lowering blood cholesterol, improving glucose tolerance, reducing average blood glucose in non-insulin-dependent diabetics, stimulation of glucose uptake by muscles, inhibition of the binding of bacteria to a host tissue, inhibition of the growth of bacteria, protection against viruses, enhancing activity of antibiotics, antifungal effects, anti-amoebic effects, prevention of tumor development, inhibition of tumor cell growth, inhibition of tumor metastasis, enhancement of natural killer cell toxicity to tumors, improvement in Alzheimer's dementia, antioxidant effects, and reaction against bacterial toxins, said dietary supplement composition comprising a nutritionally effective amount of β-glucan and at least one member selected from the group consisting of colostrum and lactoferrin.

21. The dietary supplement composition of claim 20, comprising a nutritionally effective amount of β-glucan, colostrum and lactoferrin.

22. The dietary supplement composition of claim 20, further comprising a nutritionally effective amount of citrus pectin.

23. The dietary supplement composition of claim 21, further comprising a nutritionally effective amount of citrus pectin.

24. The dietary supplement composition of claim 22, said dietary supplement composition producing in a mammal a second effect selected from the group consisting of enhancing bile acid excretion, enhancing cholesterol excretion, reducing artherosclerosis, binding heavy metals, stimulation of immune function, resistance to infection, suppression of infection, increase of tissue repair and healing, promotion of body health and athletic performance, promotion of gastrointestinal tract health, promotion of blood vessel health, promotion of glucose utilization and blood sugar balance, improved cancer inhibition, improved mental function, and improved toxin-related activities.

25. A dietary supplement composition for producing in a mammal a first effect selected from the group consisting of regulation of immune function, inhibition of cytokine release, prevention of autoimmune response from intestinal pathogens, promotion of phagocytosis by neutrophils, inhibition of mast cell enzyme involved in allergic airway response, enhancement of natural killer cell activity, anti-inflammatory effect, bioregulation during trauma stress, enhancement of hematopoietic activity, promotion of the growth of beneficial gastrointestinal bacteria, lowering blood cholesterol, improving glucose tolerance, inhibition of binding of bacteria to a host tissue, inhibition of bacteria growth, protection against viruses, enhancement of antibiotic activity, antifungal effects, prevention of tumor development, inhibition of tumor cell growth, inhibition of tumor metastases, and enhancement of natural killer cell toxicity to tumors, said dietary supplement composition comprising a nutritionally effective amount of β-glucan and lactoferrin.

26. The dietary supplement composition of claim 25, further comprising a nutritionally effective amount of citrus pectin, and producing in a mammal a second effect selected from the group consisting of stimulation of gastrointestinal tract growth, improved function of the gastrointestinal tract, enhancement of bile acid excretion, enhancement of cholesterol excretion, reduction in artherosclerosis, and binding of heavy metals.

27. A dietary supplement composition for producing in a mammal an effect selected from the group consisting of regulation of immune function, enhancement of hematopoietic activity, stimulation of gastrointestinal tract growth, improvement of gastrointestinal tract function, lowering of blood cholesterol, enhancement of bile acid excretion, enhancement of cholesterol excretion, reduction of artherosclerosis, improvement of glucose tolerance, inhibition of binding of bacteria to a host tissue, enhancement of antibiotic activity, prevention of tumor development, inhibition of tumor cell growth, inhibition of tumor metastases, enhancement of natural killer cell toxicity to tumors, and binding of heavy metals, said dietary supplement composition comprising a nutritionally effective amount of β-glucan and citrus pectin.
